(19) United States
(12) Patent Application Publication
(10) Pub. No.: US 2004/0096498 A1
(43) Pub. Date: May 20, 2004

(54) Oral pharmaceutical delivery system with improved sustained release

(76) Inventors: Alvin Kershman, Chesterfield, MO (US); Jeff L. Shear, Chesterfield, MO (US)

Correspondence Address: Greensfelder, Hernker & Gale, P.C., Intellectual Property Practice Group, Suite 2000, 10 South Broadway, St. Louis, MO, 63102, US

(21) Appl. No.: 10/425,515

(22) Filed: Apr. 29, 2003

Related U.S. Application Data
(60) Provisional application No. 60/376,387, filed on Apr. 29, 2002.

(51) Int. Cl. A61K 9/22

(52) U.S. Cl. 424/468

(57) ABSTRACT

A solid oral delivery system having improved sustained release properties made of at least one lipid, dry particles including at least one pharmaceutical, and at least one filler, wherein the dry particles are continuously coated by the lipid and form a homogeneous suspension with the lipid, wherein the suspension, when melted, exhibits thixotropic and/or pseudoplastic properties, wherein the suspension is formed into the desired dose by molding or pouring the suspension when in a liquid or semi-liquid state. The process for preparing the present delivery system by melting the lipid, blending the dry particles which include the pharmaceutical, at least one filler and, optionally, flavorings with the melted lipid, and pouring or molding the suspension to provide the solid dose, wherein the suspension, when melted, exhibits thixotropic and pseudoplastic flow properties.

### CROSS REFERENCE TO RELATED PATENTS

This utility patent application claims the benefit of Provisional Application Serial No. 60/376,387 filed Apr. 29, 2002.

### FIELD OF THE INVENTION

The present invention relates to a pharmaceutical delivery system that increases the sustained release of drugs. Said delivery system includes both human and veterinary applications. More specifically, the present invention relates to an oral pharmaceutical delivery system that is a solid lipid suspension that provides improved sustained release.

### BACKGROUND OF THE INVENTION

Drug efficacy generally depends upon the ability of the drug to reach its target in sufficient quantity to maintain therapeutic levels for the desired time period. Orally administered drugs must overcome several obstacles to reach their desired targets. Before orally administered drugs enter the general circulation of the human body, they are absorbed into the capillaries and veins of the upper gastrointestinal tract and are transported by the portal vein to the liver. The pH and enzymatic activities found in gastrointestinal fluids may inactivate the drug or cause the drug to dissolve poorly and not be absorbed. In addition, orally administered drugs are often subject to a “first pass” clearance by the liver and excreted into bile or converted into pharmacologically inactive metabolites.

The oral administration of hormones, such as testosterone or estrogen, have proven challenging. Testosterone is administered orally in a bonded form as testosterone undecanoate, methyltestosterone, or testosterone cyclodextrin, to avoid the first pass effect. When administered in a regiment of hormone replacement therapy, it is desired to have sustained release properties, yet these forms of testosterone must be taken multiple times daily.

Of particular interest is the delivery of testosterone in the unbonded form. The unbonded form of testosterone is more stable than its bonded predecessors. More of the active ingredient is delivered in a smaller dosage and tablet form. It is a simpler and less expensive manufacturing process that eliminates the additional step of bonding the testosterone. Further, the present dosage is administered with or without food, unlike the bonded form which is administered with food consumption.

“Sustained Release” generally refers to release of a drug whereby the level of drug available to the patient is maintained at some level over a desired period of time. A variety of methods and formulations are used to provide sustained release of drugs. Some of the methods are disclosed in U.S. Pat. No. 5,567,439, which is hereby incorporated by reference, which discloses controlled release systems using a shearform matrix.

The use of a lipid-based solid oral delivery system is disclosed in U.S. Pat. No. 6,340,471. U.S. Pat. No. 5,229,131 discloses a sustained release system that uses one or more individual drug-containing subunits in a unitary drug depot, such as a tablet or capsule.

None of the above-referenced patents describe the present invention as disclosed and claimed herein.

### SUMMARY OF THE INVENTION

The present invention comprises a solid oral delivery system having improved sustained release properties comprising at least one lipid, dry particles including at least one pharmaceutical, and at least one filler, wherein the dry particles are continuously coated by the lipid and form a homogeneous suspension with the lipid. The suspension, when melted, exhibits thixotropic and/or pseudoplastic properties. The suspension is formed into the desired dose by molding or pouring the suspension when in a liquid or semi-liquid state. The process for preparing the present delivery system comprises melting the lipid, blending the dry particles which include the pharmaceutical, at least one filler and, optionally, flavorings with the melted lipid, and pouring or molding the suspension to provide the solid dose. The suspension, when melted, exhibits thixotropic and pseudoplastic flow properties.

### DETAILED DESCRIPTION OF THE INVENTION

The lipids of the present invention may be of animal, vegetable or mineral origin, which are substantially water-insoluble, inert, non-toxic hydrocarbon fats and oils and derivatives thereof, and may comprise any of the commonly commercially available fats or oils approved by the Food & Drug Administration, having melting points in the range of about 90 to 160° F. (32 to 71° C.). The lipid may comprise a vegetable oil base commonly known as hard butter. Hard butters are hydrogenated, press fractionated, or other processed oils that are processed or recombined to have a solid fat index (percent solid fat vs. temperature) similar to that of cocoa butter. However, other lipids may be used that are relatively hard or solid at room temperature, but melt rapidly in the mouth at a temperature of about 92° to 98° F. (29 to 32° C.)(mouth temperature). The lipid is employed in the amounts within the range of from about 20 to 50%. Above about 50%, the suspension flows too readily and does not exhibit thixotropic or pseudoplastic flow properties. When present below about 20%, the amount of lipid is not sufficient to completely coat the dry particles.

Examples of suitable lipids include tallow, hydrogenated tallow, hydrogenated vegetable oil, almond oil, coconut oil, corn oil, cottonseed oil, light liquid petrolatum, heavy liquid petrolatum, olein, olive oil, palm oil, peanut oil, persic oil, sesame oil, soybean oil or safflower oil. Additionally, stearines can be used as a lipid in the present invention. The addition of stearines to the product provides the favorable property of mold-release. Further, the addition of stearines raises the melting point of the composition as high as about 100° F. (38° C.), which is particularly beneficial when the product is shipped or stored in unrefrigerated compartments.

The fillers of the present invention are pharmacologically inert and optionally nutritionally beneficial to humans and animals. Such fillers include cellulose such as microcrystalline cellulose, grain starches such as cornstarch, tapioca, dextrin, sugars and sugar alcohols such as sucrose sorbitol, xylitol, mannitol and the like. Preferred fillers include non-fat milk powder, whey, grain brans such as oat bran, and fruit and vegetable pulps. Preferred fillers are finely divided and have a preferred average particle size in the range of about 0.10 to 500 microns. The fillers are present in the drug delivery device in a concentration of about 50 to 80%. Optionally, the pharmaceutical particles can also serve as filler in the delivery system.

Optionally, the filler may include an emulsifier or surfactant. Any emulsifier or surfactant approved for use in foods by the Food and Drug Administration and having a relatively low HLB value, in the range of about 1 to 3, is suitable for use in the present invention. The appropriate surfactant minimizes the surface tension of the lipid, allowing it to oil wet and encapsulate the non-oil solid particles. Typically, the surfactant is present in the delivery system in the concentration of about 0.1 to 1.0%. Suitable surfactants include alkyl aryl sulfonate, alkyl sulfonates, sulfonated amides or amines, sulfated or sulfonated esters or ethers, alkyl sulfonates, of dioctyl sulfonosuccinate and the like, a hydrated aluminum silicate such as bentonite or kaolin, triglycerol monostearate, triglycerol monoshortening, monodiglyceride propylene glycol, octaglycerol monooleate, octaglyceron monostearate, and decaglycerol decaoleate. The preferred surfactant is lecithin.

In an embodiment, the pharmaceutical is microencapsulated. Such microencapsulation includes sustained release encapsulation. Any known method of encapsulation is suitable in the present invention. A preferred method involves slowly blending the drug with a filming agent solution to form granulated particles. The granulated particles are allowed to dry on a tray and are sieved to the desired size, typically in the range of from about 50 to 500 microns. In a preferred embodiment, the pharmaceutical is a mixture of encapsulated and non-encapsulated pharmaceutical. The mixture of encapsulated to non-encapsulated can be in the range of about 1:110 to 10:1.

Preferably, the pharmaceutical can be microencapsulated using methycellulose. Said microencapsulating materials are designed to release at differing pH values. The preferred pH values for controlled release are in the range of pH 4 to 7, more preferably, 5 or 6.

In another embodiment of the present invention, the pharmaceutical is not microencapsulated, but suspended in the lipid as dry particles. Typically the pharmaceutical is present in the delivery device in a concentration of 30% or less. However, the pharmaceutical can comprise all of the dried particles, acting as a filler, to provide the necessary dose.

The pharmaceuticals contemplated in the present invention are administered orally. The pharmaceuticals include drugs that have reduced bioavailability when administered orally, and drugs that do not have reduced bioavailability. Drugs that have reduced bioavailability include drugs such as analgesics, anti-inflammatory agents, gastrointestinal medications, hormone products, cardiovascular preparations, anticoagulants and antibiotics. Specific drugs include insulin, heparin, oligosaccharides, aspirin, testosterone and prednisolone. Pharmaceuticals further includes vitamins and minerals. Pharmaceuticals also includes synthetic and natural food supplements, such as glucosamine, chondroitin, bee pollen, St. John's wort, echinacea, etc. Additional pharmaceuticals are contemplated for the present invention, and are disclosed in U.S. Pat. No. 4,880,634, and U.S. Pat. No. 5,965,164, which are hereby incorporated by reference.

Optionally, the dry particles include flavorings that make the device taste and smell appealing to humans or animals. The flavorings can be natural or synthetic, and can include fruit flavorings, citrus, meat, chocolate, vanilla, fish, butter, milk, cream, egg or cheese. The flavorings are typically present in the device in the range of about 0.05 to 50.0%.

The delivery device may also include other pharmaceutically acceptable agents, such as sweetening agents, including hydrogenated starch hydrolysates, synthetic sweeteners such as sorbitol, xylitol, saccharin salts, L-aspartyl-L-phenylalanine methyl ester, as well as coloring agents, other binding agents, lubricants, such as calcium stearate, stearic acid, magnesium stearate, antioxidants such as butylated hydroxy toluene, antiflatuants such as simethicone and the like.

Optionally, rupturing agents are used to rapidly deliver the pharmaceutical into the recipient's system. A typical rupturing agent is a starch that swells in the presence of water. Various modified starches, such as carboxymethyl starch, currently marketed under the trade name Explotab or Primojel are used as rupturing agents. A preferred rupturing agent is sodium starch glycolate. When ingested, the capsule or pellet swells in the presence of gastric juices and ruptures. Preferably, the rupturing agent is present in the delivery system from about 1 to 5%.

In one embodiment of the present invention, the rupturing agent is present inside the microcapsule. As water penetrates the microcapsule, it swells the starch and ruptures the capsule, rapidly delivering the pharmaceutical to the system. Additional rupturing agents are disclosed in U.S. Pat. No. 5,567,439, which is hereby incorporated by reference.

In another embodiment, the rupturing agent is present in the lipid suspension, which ruptures the pellet, but leaves the microcapsules intact. This allows the delayed delivery of the drug farther along in the digestive system, or in the intestines. The present invention is particularly effective in this embodiment, in that the ingested pellet may be chewable, where the pellet cleaves in the lipid suspension when chewed, but leaves the microcapsules intact. Tablets or gel capsules, when chewed, typically result in damage to or rupturing of the microcapsules defeating the effectiveness of the microcapsules.

In yet another embodiment, multiple drugs have multiple encapsulations, each containing an rupturing agent. The filming agents used for encapsulation are selected to disintegrate at selected pH conditions, which rupture and release each drug at desired locations in the digestive system.

The process for preparing the above delivery system comprises melting the lipid and mixing with the surfactant. The dry particles are mixed with the melted lipid mixture to form a suspension exhibiting pseudoplastic and/or thixotropic flow properties, and poured or molded to provide solid dosage forms.

The dry particles, which include the pharmaceutical, filler and optional flavorings and additives, are pre-blended and typically have a particle size in the range of from about 50 to 500 microns. The pre-blended particles are gradually added to the heated lipid base until a high solid suspension is obtained, typically in the range of about 50 to 80% particles and from about 50 to 20% lipid.

Slow addition of the dry particles is critical in the production of the device, to insure that the particles are suspended in their micronized state and not as agglomerated clumps. Moreover, rapid addition can cause the mixing process to fail in that the melted suspension will not have the desired flow properties, but instead will be a granular oily mass (a sign of product failure). The mixing step is accomplished in a heated mixing device that insures thorough mixing of all materials with minimal shear, such as a planetary mixer or a scrape surface mixer. After the suspension is formed, the product is poured into molds and allowed to cool. De-molding and packaging are then performed. Alternatively, the suspension can be super-cooled and sheeted in a semi-soft format. The sheet is processed through forming rolls containing a design or configuration that embosses and forms the final shape.

The following examples are to illustrate the claimed invention and are not intended to limit the claims in any way. All of the percentages are by weight unless otherwise indicated.

### EXAMPLES

Example I was prepared according to the following procedure.

Forming the Suspension

The lipid (hydrogenated vegetable oil sold under the trademark KLX®) was heated in a Hobart 5 Quart planetary mixer jacketed with a heating mantle in the range of about 140 to 150° F. (60 to 66° C.) and melted. The surfactant, lecithin, was added to the lipid with mixing, and the mixture was allowed to cool to about 135° F. (° C.).

The dry particles, including the pharmaceutical (micronized, i.e., 3 to 5 microns, testosterone), the rupturing agent (sodium starch glycolate, sold under the trademark Explotab), and fillers (microcrystalline cellulose, sold under the trademark Eudragit s100, dry milk, salt and powdered sugar) were screened to a particle size in the range of about 200 and 500 microns and dry-blended. The dry particles were slowly added incrementally to the lipid/surfactant mixture with mixing over a period of about 1 hour, to provide a smooth suspension with no lumps or agglomerations. The suspension exhibited thixotropic and pseudoplastic flow properties. It was molded and cooled to about 70° F.(21° C.). The suspension shrank as it cooled, and easily released from the mold when inverted.

TABLE 1
Forming a Suspension of
Testosterone in a 250 mg Dose
BATCH FORMULA

| Ingredient | Weight (grams) | % |
|---|---|---|
| KLX (lipid) | 36.100 | 38.00 |
| Explotab (rupturing agent) | 4.750 | 5.00 |
| Eudragit s100 (cellulose) | 4.750 | 5.00 |
| Dry milk, low heat (filler) | 9.500 | 10.00 |
| Powdered sugar (filler) | 14.250 | 15.00 |
| Lecithin (surfactant) | 0.950 | 1.00 |
| Salt | 0.190 | 0.20 |
| Testosterone | 24.938 | 26.25 |
| Totals | 95 | 100.45 |

### Example 1

### Varying the Testosterone Dose 25, 50, 100, 250 mg

In Vivo Evaluation:

A study using six dogs (female beagles) was made to obtain preliminary pharmacokinetic data following a single oral dose of the delivery system. The dogs were 13-24 months old, and weighed in the range of 10.4 to 13.2 kg.

The dosing was done in four sequential one day intervals with a minimum two day rest period in between each interval. Blood was drawn immediately before the dose was administered. The results revealed minimal levels of testosterone. The animals were given the placebo or test article, as described above, at approximately the same time each day, immediately prior to being fed. The dog ate its food within 30 minutes of the dose being administered.

Blood samples were collected pre-dose and at 0.5, 1, 2, 4, 5, 6, 8 and 24 hours post dosing. At each time point, a minimum of 3 mL whole blood (or minimum volume determined by assay requirement) were collected by venipuncture of the jugular vein into non-heparinized Vacutainer tubes. The blood was centrifuged to obtain serum, which was kept on ice until placed into an appropriately sized vial, and frozen at −70° C. The samples remained frozen until delivered on dry ice to the lab for analysis. The lab used radioimmunoassay to analyse for testosterone.

Example 1 Results:

TABLE 2
Average Serum Testosterone (ng/dl)

| | Testosterone | | | |
|---|---|---|---|---|
| | Dose (mg) | | | |
| | 25 | 50 | 100 | 250 |
| | Testosterone | Testosterone | Testosterone | Testosterone |
| Time (h) | (ng/dl) | (ng/dl) | (ng/dl) | (ng/dl) |
| 0 | 0 | 1 | 0 | 26 |
| 0.5 | 286 | 154 | 270 | 264 |
| 1 | 390 | 286 | 309 | 555 |
| 2 | 425 | 376 | 450 | 835 |
| 4 | 118 | 288 | 522 | 1032 |
| 5 | 35 | 215 | 618 | 829 |
| 6 | 53 | 107 | 357 | 980 |
| 8 | 23 | 54 | 422 | 757 |
| 24 | 1 | 7 | 2 | 8 |

Determining the sustained release properties of the samples in Example 1 by evaluating the amount of time the blood serum levels fell between about 300 and 1100 ng/dl, the sustained release times for Example 1 are given in Table 3. It is desirable to have release properties that are fairly constant with time. It is undesirable to have sharp peaks or drops in serum concentration of the drug.

### Sustained Release Times

### Example 1

TABLE 3

| Testosterone Dose (mg) | Lipid Suspension Time (h) |
|---|---|
| 25 | 1 |
| 50 | 0 |
| 100 | 7 |
| 250 | 7 |

Longer sustained release times are noted for doses of 100 mg and higher. Smaller doses fail to maintain the desired levels for a sufficient length of time. It is important that the present data is taken using dogs as test animals. It is generally recognized that the metabolism of dogs is higher than that of humans, and that humans will typically display higher blood serum levels for a greater period of time under similar test conditions. It is expected that humans will experience even greater sustained release levels than those shown in the dogs.

### Example 2

### Varying the Amount of Rupturing Agent

Samples of a lipid suspension were prepared as in Example 1, wherein the amount of testosterone administered was 250 mg, and the amount of rupturing agent was varied as follows: 0, 1, 2 and 5%.

In Vivo Evaluation:

A study using four dogs (female beagles) was made to obtain preliminary pharmacokinetic data following a single oral dose of the delivery system. The dogs were 13-24 months old, and weighed in the range of 11.1 to 12.6 kg.

The dosing was done in four sequential one day intervals with a minimum four day rest period in between each interval. Blood was drawn immediately before the dose was administered. The results revealed minimal levels of testosterone. The animals were given the placebo or test article, as described above, at approximately the same time each day, immediately prior to being fed. The dog ate its food within 30 minutes of the dose being administered.

Blood samples were collected pre-dose and at 3, 6, 8, 10, 12, 16, 20 and 24 hours post dosing. At each time point, a minimum of 3 mL whole blood (or minimum volume determined by assay requirement) were collected by venipuncture of the jugular vein into non-heparinized Vacutainer tubes. The blood was centrifuged to obtain serum, which was kept on ice until placed into an appropriately sized vial, and frozen at −70° C. The samples remained frozen until delivered on dry ice to the lab for analysis. The lab used radioimmunoassay to analyse for testosterone.

Test Results:

TABLE 4
Average Serum Testosterone (ng/dl)

% Explotab*

| Time (h) | 5 | 0 | 1 | 2 |
|---|---|---|---|---|
| 0 | 2.0 | 0.0 | 2.5 | 0.3 |
| 3 | 433.5 | 485.8 | 274.0 | 690.8 |
| 6 | 1257.0 | 537.3 | 561.3 | 920.0 |
| 8 | 479.8 | 520.8 | 772.5 | 776.0 |
| 10 | 330.3 | 410.5 | 553.3 | 840.0 |
| 12 | 224.5 | 243.5 | 449.3 | 293.8 |
| 16 | 31.5 | 213.0 | 212.8 | 61.3 |
| 20 | 12.0 | 72.3 | 88.0 | 29.0 |
| 24 | 6.8 | 48.3 | 54.5 | 27.3 |

*The rupturing agent.

Each dose, for a period of time, is above 300 ng/dl average serum testosterone. The samples in Example 2 demonstrate improved sustained release properties, maintaining the desired levels of serum testosterone from about 6 to 7 h. The sample with 5% Explotab had one serum level of testosterone exceeding 1100 ng/dl. It is desirable to have sustained release properties without the spike observed in the sample with 5% Explotab.

### Example 3

Varying the Surfactant

An in vivo evaluation, of the present invention was made, using the formulation from Table 1, but varying the surfactant as follows. The same procedure was followed as described in Example 3, except that three dogs were used and there was a two day washout.

TABLE 5
Average Serum Testosterone (ng/dl)

| | Surfactant | | |
|---|---|---|---|
| Time (h) | Lecithin | No Surfactant | Durem 300* |
| 0 | 0.8 | 0.0 | 0.0 |
| 0.5 | 81.6 | 94.3 | 104.7 |
| 1.0 | 395.8 | 277.3 | 217.7 |
| 2.0 | 904.8 | 609.7 | 1136.7 |
| 4.0 | 1347.6 | 1410.0 | 581.0 |
| 5.0 | 1298.4 | 702.3 | 591.0 |
| 6.0 | 824.8 | 632.7 | 688.7 |
| 8.0 | 430.4 | 375.0 | 576.3 |
| 24.0 | 8.6 | 48.0 | 59.3 |

*Monodiglyceride propylene glycol surfactant.

All of the samples gave the testosterone levels above 300 ng/dl for about 6 h or more. However, each sample had one or two serum testosterone levels exceeding 1100 ng/dl. Lower dosages may be indicated, as it is undesirable to have serum levels above 1100 ng/dl.

### Example 4

### 100 mg Microencapsulated, 150 mg Micronized Testosterone Combined for 250 mg Dose

Four delivery systems of testosterone were prepared. Three samples contained microencapsulated micronized testosterone (100 mg). The three samples were microencapsulated with methylcellulose designed to release at either pH 5, 6 or 7. The remaining 150 mg of testosterone was micronized. The fourth sample was prepared with un-encapsulated testosterone. The four samples were formulated into a lipid suspension as disclosed in Example 1 and given to four dogs. Serum levels of testosterone were measured as in Example 1.

TABLE 6
Serum Levels of Testosterone (ng/dl)

| Time (h) | Un-encapsulated | pH 5 Release | pH 6 Release | pH 7 Release |
|---|---|---|---|---|
| 0 | 79 | 5 | 12 | 15 |
| 1.5 | 438 | 166 | 314 | 254 |
| 3 | 649 | 179 | 333 | 290 |
| 6 | 603 | 426 | 487 | 271 |
| 9 | 302 | 438 | 599 | 348 |
| 12 | 147 | 576 | 377 | 344 |
| 15 | 52 | 351 | 266 | 195 |
| 18 | 25 | 86 | 90 | 173 |
| 21 | 18 | 55 | 75 | 190 |
| 24 | 16 | 30 | 112 | 117 |

The un-encapsulated sample provides some sustained release properties, but not as great as those of the pH 5 and pH 6 microencapsulated release samples. The pH 7 microencapsulated release sample provided a much lower release time, as though a much smaller dose of testosterone was administered. All of the samples in Table 6 gave consistent sustained release properties without any spikes above 700 ng/dl.

TABLE 7
Sustained Release Times
Partial Microencapsulation

| Testosterone Form | Sustained Release Time (h) |
|---|---|
| Un-encapsulated | 7.5 |
| pH 5 Release | 9 |
| pH 6 Release | 10.5 |
| pH 7 Release | 3 |

1. A sustained release solid oral delivery system comprising at least one lipid, dry particles including at least one pharmaceutical, and at least one filler, wherein the dry particles are continuously coated by the lipid and form a homogeneous suspension with the lipid, wherein the suspension, when melted, exhibits thixotropic and/or pseudoplastic properties, wherein the suspension is formed into the desired dose by molding or pouring the suspension when in a liquid or semi-liquid state, and wherein the system displays improved sustained release properties.

2. The pharmaceutical delivery system of claim 1, wherein at least part of the pharmaceutical is microencapsulated.

3. The pharmaceutical delivery system of claim 1, wherein the dry particles include a rupturing agent.

4. The pharmaceutical delivery system of claim 2, wherein the dry particles include a rupturing agent.

5. The pharmaceutical delivery system of claim 2, wherein all of the pharmaceutical is micro encapsulated.

6. The pharmaceutical delivery system of claim 2, wherein the ratio of encapsulated to non-encapsulated is in the range of 1:10 to 10:1.

7. The pharmaceutical delivery system of claim 2, wherein the microencapsulating film releases at a pH in the range of about 4 to 7.

8. The pharmaceutical delivery system of claim 2, wherein the microencapsulating film releases at a pH in the range of about 5 to 6.

9. The pharmaceutical delivery system of claim 3, wherein the rupturing agent comprises sodium starch glycolate.

10. The pharmaceutical delivery system of claim 1, wherein the lipid includes a surfactant.

11. The pharmaceutical delivery system of claim 10, wherein the surfactant comprises lecithin.

12. A sustained release solid oral delivery system comprising
a) at least one lipid,
b) dry particles including at least one pharmaceutical, and
c) at least one filler,
wherein at least part of the pharmaceutical is microencapsulated,
wherein the dry particles are continuously coated by the lipid and form a homogeneous suspension with the lipid,
wherein the suspension, when melted, exhibits thixotropic and/or pseudoplastic properties,
wherein the suspension is formed into the desired dose by molding or pouring the suspension when in a liquid or semi-liquid state, and
wherein the system displays improved sustained release properties.

13. The pharmaceutical delivery system of claim 12, wherein the dry particles include a rupturing agent.

14. The pharmaceutical delivery system of claim 12, wherein all of the pharmaceutical is microencapsulated.

15. The pharmaceutical delivery system of claim 12, wherein the ratio of encapsulated to non-encapsulated is in the range of 1:10 to 10:1.

16. The pharmaceutical delivery system of claim 12, wherein the microencapsulating film releases at a pH in the range of about 4 to 7.

17. The pharmaceutical delivery system of claim 12, wherein the microencapsulating film releases at a pH in the range of about 5 to 6.

18. The pharmaceutical delivery system of claim 13, wherein the rupturing agent comprises sodium starch glycolate.

19. The pharmaceutical delivery system of claim 12, wherein the lipid includes a surfactant.

20. The pharmaceutical delivery system of claim 19, wherein the surfactant comprises lecithin.

21. The pharmaceutical delivery system of claim 1, wherein said pharmaceutical is selected form the group consisting of analgesics, antibodies, anti-inflammatory agents, cardiovascular drugs, gastrointestinal medicines, hormones and laxatives.

22. The pharmaceutical delivery system of claim 12, wherein said pharmaceutical is selected form the group consisting of analgesics, antibodies, anti-inflammatory agents, cardiovascular drugs, gastrointestinal medicines, hormones and laxatives.

23. A method for preparing a sustained release solid oral delivery system comprising melting at least one lipid, blending dry particles which include at least one pharmaceutical and at least one filler, and pouring or molding the suspension to provide a solid dose, wherein the suspension, when melted, exhibits thixotropic and pseudoplastic flow properties, and wherein the delivery system displays improved sustained release properties.

24. The method of claim 23, wherein at least part of the pharmaceutical is microencapsulated.

25. The method of claim 23, wherein the dry particles include a rupturing agent.

26. The method of claim 24, wherein the dry particles include a rupturing agent.

27. The method of claim 24, wherein all of the pharmaceutical is microencapsulated.

28. The method of claim 24, wherein the ratio of encapsulated to non-encapsulated is in the range of 1:10 to 10:1.

29. The method of claim 24, wherein the microencapsulating film releases at a pH in the range of about 4 to 7.

30. The method of claim 24, wherein the microencapsulating film releases at a pH in the range of about 5 to 6.

31. The method of claim 25, wherein the rupturing agent comprises sodium starch glycolate.

32. The method of claim 23, wherein the lipid includes a surfactant.

33. The method of claim 32, wherein the surfactant comprises lecithin.

34. A method of preparing a sustained release solid oral delivery system comprising
a) microencapsulating at least part of a pharmaceutical,
b) melting at least one lipid,
c) dry-mixing dry particles including the pharmaceutical, and at least one filler,
d) mixing the dry particle mixture with the melted lipid to form a suspension,
wherein the dry particles are continuously coated by the lipid and form a homogeneous suspension with the lipid,
wherein the suspension, when melted, exhibits thixotropic and/or pseudoplastic properties,
wherein the suspension is formed into the desired dose by molding or pouring the suspension when in a liquid or semi-liquid state, and
wherein the system displays improved sustained release properties.

35. The method of claim 34, wherein the dry particles include a rupturing agent.

36. The method of claim 34, wherein all of the pharmaceutical is microencapsulated.

37. The method of claim 34, wherein the ratio of encapsulated to non-encapsulated is in the range of 1:10 to 10:1.

38. The method of claim 34, wherein the microencapsulating film releases at a pH in the range of about 4 to 7.

39. The method of claim 34, wherein the microencapsulating film releases at a pH in the range of about 5 to 6.

40. The method of claim 35, wherein the rupturing agent comprises sodium starch glycolate.

41. The method of claim 34, wherein the lipid includes a surfactant.

42. The method of claim 41, wherein the surfactant comprises lecithin.

43. The method of claim 23, wherein said pharmaceutical is selected form the group consisting of analgesics, antibodies, anti-inflammatory agents, cardiovascular drugs, gastrointestinal medicines, hormones and laxatives.

44. The method of claim 34, wherein said pharmaceutical is selected form the group consisting of analgesics, antibodies, anti-inflammatory agents, cardiovascular drugs, gastrointestinal medicines, hormones and laxatives.
